(19) United States
(12) Patent Application Publication
(10) Pub. No.: US 2021/0401511 A1
(43) Pub. Date: Dec. 30, 2021

(54) SURGERY SUPPORT SYSTEM AND SURGERY SUPPORT METHOD

(71) Applicant: CANON MEDICAL SYSTEMS CORPORATION, Otawara-shi (JP)

(72) Inventors: Mitsuo SEKINE, Nasushiobara (JP); Sayaka TAKAHASHI, Nasushiobara (JP)

(73) Assignee: CANON MEDICAL SYSTEMS CORPORATION, Otawara-shi (JP)

(21) Appl. No.: 17/304,969

(22) Filed: Jun. 29, 2021

(30) Foreign Application Priority Data
Jun. 29, 2020 (JP) 2020-111615

(51) Int. Cl. A61B 34/00; A61B 90/00

(52) U.S. Cl. CPC A61B 34/25; A61B 2090/372; A61B 90/37

(57) ABSTRACT

A surgery support system according to an embodiment includes processing circuitry. The processing circuitry acquires medical information of a subject under surgery. The processing circuitry detects an event relating to an abnormality, based on the acquired medical information of the subject. The processing circuitry associates a point of time of detecting the event relating to the abnormality with the medical information acquired at the point of time to generate association information.

### CROSS-REFERENCE TO RELATED APPLICATIONS

This application is based upon and claims the benefit of priority from Japanese Patent Application No. 2020-111615, filed on Jun. 29, 2020; the entire contents of which are incorporated herein by reference.

### FIELD

Embodiments disclosed in the present specification and the drawings relate generally to a surgery support system and a surgery support method.

### BACKGROUND

Conventionally, various surgery support systems are known and used during surgery. For example, in the field of laparoscopic surgery, such surgery support systems are known that each generate, in order to avoid blood vessels and organs from being damaged, a virtual endoscopic image from a computed tomography (CT) image captured before surgery. The surgery support system then presents, during surgery, the generated virtual endoscopic image in conjunction with an actual endoscopic image.

### BRIEF DESCRIPTION OF THE DRAWINGS

FIG. 1 is a view illustrating an example of a configuration of a surgery support system according to a first embodiment;

FIG. 2 is a view illustrating an example of display control executed by a control function according to the first embodiment;

FIG. 3 is a view illustrating an example of the display control executed by the control function according to the first embodiment;

FIG. 4 is a view illustrating an example of the display control executed by the control function according to the first embodiment; and

FIG. 5 is a flowchart for describing steps of processing executed by a surgery support apparatus according to the first embodiment.

### DETAILED DESCRIPTION

According to an embodiment, a surgery support system comprises processing circuitry. The processing circuitry is configured to acquire medical information of a subject under surgery. The processing circuitry is configured to detect an event relating to an abnormality, based on the acquired medical information of the subject. The processing circuitry is configured to associate a point of time of detecting the event relating to the abnormality with the medical information acquired at the point of time to generate association information.

Embodiments of a surgery support system and a surgery support method will now be described in detail with reference to the accompanying drawings. Note that the surgery support system and the surgery support method according to the present application are not limited to the embodiments described below. Furthermore, it is possible to combine the embodiments with other embodiments and prior arts within a range in which no inconsistency arises in processing contents.

### First Embodiment

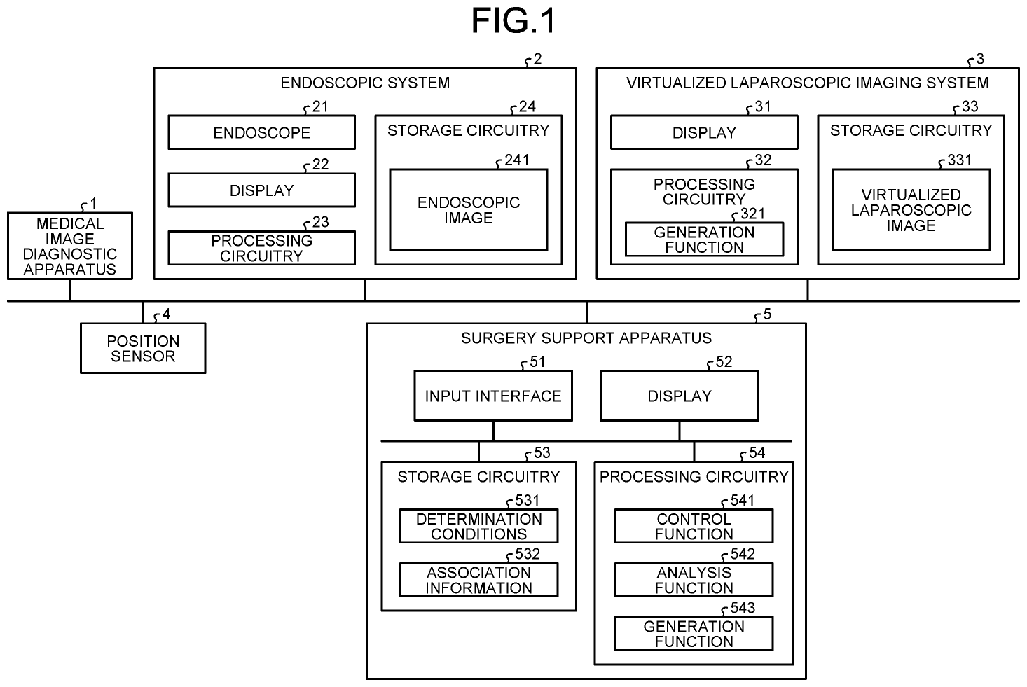

FIG. 1 is a view illustrating an example of a configuration of a surgery support system 10 according to a first embodiment. Note herein that, in FIG. 1, the surgery support system 10 including a surgery support apparatus configured to execute surgery support according to the present application is described. However, embodiments are not limited to the following description. A surgery support method described below may be executed by any apparatus in the surgery support system 10.

For example, as illustrated in FIG. 1, the surgery support system 10 according to the present embodiment includes a medical image diagnostic apparatus 1, an endoscopic system 2, a virtualized laparoscopic imaging system 3, a position sensor 4, and a surgery support apparatus 5. Note herein that the apparatuses and the systems are communicably coupled to each other via a network. Furthermore, in the first embodiment, a case in which laparoscopic surgery is performed is described as example surgery. However, the type of surgery is not limited to this laparoscopic surgery. The first embodiment may be applied to other types of surgery. Furthermore, the surgery support system 10 may include another system (e.g., a hospital information system (HIS)) and another apparatus (e.g., an image keeping apparatus) than those illustrated in the drawings.

The medical image diagnostic apparatus 1 is configured to capture and collect medical images of a subject. The medical image diagnostic apparatus 1 then sends the collected medical images to the virtualized laparoscopic imaging system 3 and the surgery support apparatus 5, for example. For example, the medical image diagnostic apparatus 1 is an X-ray diagnostic apparatus, an X-ray computed tomography (CT) apparatus, a magnetic resonance imaging (MRI) apparatus, an ultrasonic diagnostic apparatus, a single photon emission computed tomography (SPECT) apparatus, or a positron emission computed tomography (PET) apparatus.

The medical image diagnostic apparatus 1 collects medical images regarding the subject who undergoes surgery. Specifically, the medical image diagnostic apparatus 1 collects, before and after surgery, medical images of a portion subjected to surgery. The medical image diagnostic apparatus 1 then sends the collected medical images to the virtualized laparoscopic imaging system 3 and the surgery support apparatus 5, for example.

The endoscopic system 2 includes an endoscope 21, a display 22, processing circuitry 23, and storage circuitry 24. The endoscope 21 has an insertion part to be inserted into the subject and an operation part configured to operate the insertion part. The insertion part represents a treatment part configured to treat a target portion (an involved part) subjected to surgery inside the subject and an imaging part configured to capture images inside the subject. The operation part accepts an operation of a medical operator operating the treatment part and the imaging part.

The treatment part is forceps, an electrosurgical knife, or a suturing device, for example. Furthermore, the imaging part has an imaging element, such as a charge coupled device (CCD) type image sensor or a complementary metal oxide semiconductor (CMOS) image sensor, a lens, and a light emitting part. The imaging part is configured to cause the imaging element to capture images of the involved part to which light is emitted from the light emitting part.

The display 22 is configured to display each of the images (endoscopic images) captured by the imaging part. The processing circuitry 23 is coupled to the endoscope 21, the display 22, and the storage circuitry 24. The processing circuitry 23 is configured to wholly control the endoscopic system. For example, the processing circuitry 23 controls operating of the treatment part and collecting of endoscopic images by the imaging part in the endoscope 21, displaying of the endoscopic images by the display 22, and storing of the endoscopic images in the storage circuitry 24. The storage circuitry 24 is configured to store endoscopic images 241 collected by the imaging part of the endoscope 21.

For example, the medical operator such as a medical doctor inserts, into the subject, the endoscope 21 having, as the insertion part, the treatment part serving as forceps or an electrosurgical knife, for example, and the endoscope 21 having, as the insertion part, the imaging part. The medical operator then, while observing an endoscopic image collected by the imaging part and displayed on the display 22, operates the treatment part to treat the target portion (the involved part) subjected to surgery inside the subject.

The virtualized laparoscopic imaging system 3 includes a display 31, processing circuitry 32, and storage circuitry 33. The display 31 is configured to display an image generated by the processing circuitry 32. Specifically, the display 31 displays a virtualized laparoscopic image generated on the basis of a medical image collected by the medical image diagnostic apparatus 1.

The processing circuitry 32 is coupled to the display 31 and the storage circuitry 33. The processing circuitry 32 is configured to wholly control the virtualized laparoscopic imaging system. Specifically, the processing circuitry 32 controls acquiring of medical images from the medical image diagnostic apparatus 1, displaying of virtualized laparoscopic images by the display 22, and storing of the virtualized laparoscopic images in the storage circuitry 33, for example. Furthermore, the processing circuitry 32 executes a generation function 321 to generate a virtualized laparoscopic image using a medical image. For example, the generation function 321 is configured to generate a virtualized laparoscopic image on the basis of a three-dimensional CT image that the X-ray CT apparatus serving as the medical image diagnostic apparatus 1 collected before surgery from an abdominal part of the subject.

In an example case, the generation function 321 generates a virtualized laparoscopic image of the inside of the abdominal cavity, that is, a projection image in a predetermined sight direction, on the basis of information of a portion inside the abdominal cavity in a two-dimensional CT image generated by using a three-dimensional CT image. The storage circuitry 33 is configured to store a virtualized laparoscopic image 331 generated by the processing circuitry 32.

The position sensor 4 has a sensor part, a magnetic field generation part, and a signal reception part. The sensor part is a magnetic sensor, for example. The sensor part is disposed at a tip of the insertion part in the endoscope 21 or inside the subject. The magnetic field generation part is disposed adjacent to the subject. The magnetic field generation part is configured to outwardly form a magnetic field around the part itself. The signal reception part is configured to receive a signal outputted by the sensor part.

The sensor part is configured to detect a three-dimensional magnetic field formed by the magnetic field generation part. The sensor part then calculates, on the basis of information of the detected three-dimensional magnetic field, its position information (coordinates and an angle) in a space where the magnetic field generation part serves as an origin. The sensor part thus sends its calculated position information to the signal reception part. For example, the position information received from the sensor part attached at the tip of the insertion part in the endoscope 21 is indicative of the position of the tip of the insertion part in the space where the magnetic field generation part serves as the origin. Furthermore, the position information received from the sensor part disposed inside the subject (e.g., the organ regarded as the involved part) is indicative of the position of the involved part in the space where the magnetic field generation part serves as the origin. The signal reception part sends the position information received from the sensor part to the virtualized laparoscopic imaging system 3 and the surgery support apparatus 5.

Note herein that the virtualized laparoscopic imaging system 3 is able to use the position information acquired by the position sensor 4 described above to generate and display a virtualized laparoscopic image in conjunction with an endoscopic image. In such a case, three-dimensional coordinates in the space where the magnetic field generation part serves as the origin are first aligned with three-dimensional coordinates in a three-dimensional medical image used to generate a virtualized laparoscopic image.

For example, the generation function 321 extracts the position (the position of the organ, at which the sensor part is disposed), in the three-dimensional medical image, corresponding to the position information acquired by the sensor part disposed in the subject (e.g., the organ regarded as the involved part). The generation function 321 then aligns, at the same position, the extracted position with the position acquired by the sensor part. Note herein that the generation function 321 executes an alignment as described above on the basis of the position information from the sensor part disposed at each of a plurality of positions in the subject. The generation function 321 thus aligns three-dimensional coordinates in the space where the magnetic field generation part serves as the origin with three-dimensional coordinates in a three-dimensional medical image used to generate a virtualized laparoscopic image.

Note that a method for executing an alignment of three-dimensional coordinates in the space where the magnetic field generation part serves as the origin with three-dimensional coordinates in a three-dimensional medical image used to generate a virtualized laparoscopic image is not limited to the method described above. Another method may be used. For example, position information of a portion in a subject, when the portion is depicted in an endoscopic image captured by the imaging part, to which the sensor part is attached, of the endoscope 21 may be used.

In such a case, for example, the generation function 321 calculates, on the basis of position information from the sensor part attached to the imaging part, the three-dimensional coordinates of a portion (e.g., a distinctive portion) depicted in an endoscopic image, in the space where the magnetic field generation part serves as the origin. The generation function 321 then extracts the position, in the three-dimensional medical image, corresponding to the portion for which the three-dimensional coordinates have been calculated. The generation function 321 thus aligns, at the same position, the extracted position with the position at which the three-dimensional coordinates have been calculated. Note herein that the generation function 321 executes an alignment as described above for each of a plurality of positions in the subject. The generation function 321 thus aligns three-dimensional coordinates in the space where the magnetic field generation part serves as the origin with three-dimensional coordinates in a three-dimensional medical image used to generate a virtualized laparoscopic image.

As described above, when an alignment is executed, the generation function 321 generates, on the basis of the position information of the sensor part attached to the imaging part of the endoscope 21, a virtualized laparoscopic image in conjunction with an endoscopic image. For example, the generation function 321 generates a virtualized laparoscopic image, that is, a projection image of a three-dimensional CT image (the inside of the abdominal cavity) from a point of sight that is the three-dimensional coordinates acquired by the sensor part attached to the imaging part of the endoscope 21 in a projection direction that is the imaging direction derived from the angle of the sensor part.

The generation function 321 then sequentially generates virtualized laparoscopic images from the varying point of sight in the varying projection direction in accordance with changes in position of the imaging part (changes in three-dimensional coordinates acquired by and the angle of the sensor part). As described above, as the virtualized laparoscopic images sequentially generated are sequentially displayed, the virtualized laparoscopic images are displayed in conjunction with changes in the endoscopic images.

Note herein that the generation function 321 is able to use the position information acquired by the sensor part disposed in the subject (e.g., the organ regarded as the involved part) to reflect, in a virtualized laparoscopic image, a change in shape of the organ subjected to surgery. For example, the generation function 321 calculates an amount of change each time there is a change of position information acquired by the sensor part disposed in the organ. The generation function 321 then adds the change at the calculated amount of change to a corresponding position in a three-dimensional medical image. The generation function 321 then uses the medical image after changed to generate a virtualized laparoscopic image. The generation function 321 thus reflects the change in shape of the organ subjected to surgery in the virtualized laparoscopic image.

The surgery support apparatus 5 is configured to generate information regarding an event relating to an abnormality, when the event occurred during surgery, on the basis of various types of information acquired during the surgery. Specifically, the surgery support apparatus 5 acquires information from the medical imaging apparatus and various types of medical devices during surgery. The surgery support apparatus 5 then generates information regarding an event relating to an abnormality during surgery, on the basis of the acquired information. For example, the surgery support apparatus 5 is implemented by a computer apparatus such as a work station, a personal computer, or a tablet terminal.

For example, the surgery support apparatus 5 includes an input interface 51, a display 52, storage circuitry 53, and processing circuitry 54. The surgery support apparatus 5 is then coupled, via the network, to the medical image diagnostic apparatus 1, the endoscopic system 2, the virtualized laparoscopic imaging system 3, and the position sensor 4.

The input interface 51 is configured to accept input operations of various types of instructions and various types of information from a user. Specifically, the input interface 51 is coupled to the processing circuitry 54. The input interface 51 converts an input operation accepted from the user into an electric signal, and outputs the electric signal to the processing circuitry 54. For example, the input interface 51 is implemented by a trackball, switch buttons, a mouse, a keyboard, a touch pad having an operation face to be touched for an input operation, a touch screen in which a display screen and a touch pad are integrated with each other, a non-contact input interface using an optical sensor, and voice input interface, for example. Note that, in the present specification, the input interface 51 is not limited to those that include physical operation parts such as a mouse and a keyboard. For example, examples of the input interface 51 include a processing circuit for electric signals. The processing circuit is configured to receive an electric signal corresponding to an input operation, from an external input device provided separately from the present device, and to output the electric signal to a control circuit.

The display 52 is configured to display various types of information and various types of data. Specifically, the display 52 is coupled to the processing circuitry 54. The display 52 thus displays various types of information and various types of data outputted from the processing circuitry 54. For example, the display 52 is implemented by a liquid crystal display, a cathode ray tube (CRT) display, an organic electro-luminescence (EL) display, a plasma display, or a touch panel.

The storage circuitry 53 is configured to store various types of data and various types of computer programs. Specifically, the storage circuitry 53 is coupled to the processing circuitry 54. The storage circuitry 53 stores data inputted from the processing circuitry 54 or read and output stored data to the processing circuitry 54. For example, the storage circuitry 53 is implemented by a semiconductor memory element, such as a random access memory (RAM) or a flash memory, or a hard disk or an optical disk.

For example, the storage circuitry 53 stores determination conditions 531 and association information 532. Note that the determination conditions 531 and the association information 532 will be described later in detail.

The processing circuitry 54 is configured to wholly control the surgery support apparatus 5. For example, the processing circuitry 54 performs various types of processing in accordance with an input operation accepted from the user via the input interface 51. For example, the processing circuitry 54 causes the storage circuitry 53 to store data sent from other devices. Furthermore, for example, the processing circuitry 54 outputs data read from the storage circuitry 53 and sends the data to other devices. Furthermore, for example, the processing circuitry 54 causes the display 52 to display data read from the storage circuitry 53.

Note herein that the processing circuitry in the endoscopic system 2, the virtualized laparoscopic imaging system 3, and the surgery support apparatus 5 described above are implemented by processors, for example. In such a case, the processing functions described above are stored in the storage circuitry in the form of computer programs that are executable by a computer. The processing circuitry then read and execute the computer programs stored in the storage circuitry to implement functions corresponding to the computer programs. In other words, when the processing circuitry read the computer programs, the processing circuitry have the processing functions illustrated in FIG. 1.

Note that the processing circuitry may include a plurality of independent processors in a combined manner. The processors may respectively execute the computer programs to implement the processing functions. Furthermore, the processing functions that the processing circuitry have may be implemented appropriately in an integrated manner into a single processing circuit or in a dispersed manner among a plurality of processing circuits. Furthermore, the processing functions that the processing circuitry have may be implemented by hardware and software in a mixed manner in circuits, for example. Furthermore, the example case in which the computer programs corresponding to the processing functions are stored in the single storage circuit has been described in here. However, embodiments are not limited to the example case. For example, the computer programs corresponding to the processing functions may be stored in a plurality of storage circuits in a dispersed manner. The processing circuits may be configured to read and execute the computer programs from the storage circuits.

The example of the configuration of the surgery support system 10 according to the present embodiment has been described above. For example, the surgery support system 10 according to the present embodiment is installed in an operation room of a medical facility such as a hospital or a medical office. The surgery support system 10 is used to support a confirmation regarding an abnormality occurred during surgery performed by a user such as a medical doctor.

For example, in the field of laparoscopic surgery, there may be bleeding unnoticed by a medical operator. In example cases, a device is in contact with a blood vessel, a blood vessel behind a membrane is damaged when the membrane is peeled off with an electrosurgical knife, there is a rapture at another pressurized location than a location where a force of forceps holding an organ increases, and there is a rapture at another weakened location than a location where forceps holding a blood vessel is pulled a little more forcefully.

When such bleeding occurs, a location of the bleeding needs to be identified to arrest the bleeding in order to continue endoscopic surgery. However, the bleeding may hide the field of view of the endoscope, making it difficult to identify the location of the bleeding. Furthermore, the medical operator may view backward endoscopic images to check and search for a location of bleeding. However, the medical operator may not know when and where the medical operator did an involving operation. Therefore, the medical operator may face difficulties in identifying the location of bleeding from the endoscopic images. Otherwise, even when the medical operator is finally able to identify the location of bleeding, the medical operator may have to spend much time. Furthermore, with a virtual endoscopic image, a medical operator is able to know the structure of organs in a body of a subject, even when the structure is outside the field of view of an endoscope. However, such a virtual endoscopic image does not reflect an abnormality, such as a bleeding event, occurred during surgery. Therefore, the medical operator faces difficulties in identifying such an abnormality through a virtual endoscopic image.

As described above, when the medical operator has to spend much time to identify a location of bleeding, or when the medical operator is not able to arrest the bleeding, the medical operator may make a shift to laparotomy to continue the surgery. However, when a cut may expand, or when the medical operator has failed to promptly make the shift to laparotomy, the patient may fall into a critical condition, causing the patient to bear a large burden.

To address this issue, the surgery support apparatus 5 in the surgery support system 10 according to the present embodiment is configured to acquire various types of information during surgery. The surgery support apparatus 5 then associates the acquired information with information of a time to generate association information, allowing an easy confirmation of information regarding an abnormality occurred during the surgery.

Specifically, the surgery support apparatus 5 continuously acquires information from the medical image diagnostic apparatus 1, the endoscopic system 2, and other various types of medical devices used during surgery. The surgery support apparatus 5 then analyzes the acquired information, determines an event relating to an abnormality, associates information regarding the event with a point of time of acquiring the information, and stores the information and the point of time of time associated with each other. Therefore, the surgery support system 10 is able to determine that, when an event (an abnormality) such as bleeding occurs during surgery, there is the event relating to the abnormality, and to present information regarding the event, allowing an easy confirmation of the information regarding the abnormality occurred during the surgery. The surgery support apparatus 5 having the configuration as described above will now be described in detail.

For example, as illustrated in FIG. 1, in the present embodiment, the processing circuitry 54 of the surgery support apparatus 5 executes a control function 541, an analysis function 542, and a generation function 543. Note herein that the control function 541 is an example of an acquisition unit and a display control unit. Furthermore, the analysis function 542 is an example of a detection unit. Furthermore, the generation function 543 is an example of a generation unit.

The control function 541 is configured to acquire various types of data (medical information) from the other apparatuses coupled via the network, and to cause the storage circuitry 53 to store the acquired medical information. For example, the control function 541 acquires medical images collected by the medical image diagnostic apparatus 1, endoscopic images generated by the endoscopic system 2, and virtualized laparoscopic images generated by the virtualized laparoscopic imaging system 3.

Note herein that the control function 541 is able to acquire medical information of the subject before and during surgery. For example, the control function 541 acquires medical images collected before surgery and medical images collected during surgery. Furthermore, the control function 541 acquires endoscopic images during surgery. Furthermore, the control function 541 acquires virtualized laparoscopic images generated before surgery and virtualized laparoscopic images generated during surgery.

Furthermore, the control function 541 is able to acquire various types of medical information from the subject under surgery. For example, the control function 541 acquires vital information from the subject under surgery, and position information acquired by the position sensor 4 or various types of information acquired by various types of sensors attached to the endoscope 21. Note herein that examples of the various types of sensors attached to the endoscope 21 include a micro-electro-mechanical systems (MEMS) sensor configured to acquire pressure information of forceps. When a MEMS sensor as described above is attached to the endoscope 21, the control function 541 is also able to acquire pressure information acquired by the MEMS sensor.

Furthermore, the control function 541 causes the display 52 to display various types of medical information. For example, the control function 541 causes the display 52 to display information generated on the basis of a result of analysis of the acquired medical information. Note herein that the control function 541 is also able to send the generated information to the other apparatuses via the network to control and cause the displays of the other devices to display the generated information. For example, the control function 541 sends the generated information to the endoscopic system 2 and the virtualized laparoscopic imaging system 3. The endoscopic system 2 and the virtualized laparoscopic imaging system 3 each cause its display to display the information received from the surgery support apparatus 5.

The analysis function 542 is configured to detect an event relating to an abnormality, on the basis of the acquired medical information of the subject. Specifically, the analysis function 542 compares the medical information acquired by the control function 541 with the determination conditions 531 that the storage circuitry 53 has stored. The analysis function 542 then detects information conforming to any one of the determination conditions, in the acquired medical information, as an event relating to an abnormality. For example, the analysis function 542 detects an event relating to an abnormality during surgery, on the basis of medical images acquired from the medical image diagnostic apparatus 1, endoscopic images acquired from the endoscopic system 2, and the vital information, for example. In an example case, the analysis function 542 is able to detect an event relating to an abnormality, on the basis of an image feature amount in an image (a medical image or an endoscopic image) of the subject, when the image is acquired during surgery. Note that the determination conditions 531 include various types of conditions corresponding to medical information to be acquired.

The analysis function 542 detects, as an event relating to an abnormality, an inducement event that induces an abnormality, for example. In such a case, for example, determination conditions corresponding to an analysis using an endoscopic image include conditions such as “whether the treatment part is in contact with a blood vessel”, “the degree of penetration of an electrosurgical knife into tissue”, “a period of time during which a blood vessel is pinched with forceps”, “a period of time during which an organ is held with forceps”, and “the degree of deformation of an organ”.

For example, the condition “whether the treatment part is in contact with a blood vessel” in the determination conditions 531 represents a condition of whether the treatment part of the endoscope 21 is in contact with a blood vessel, and further represents a condition in which, when there is a contact, the degree of the contact is classified in a stepwise manner. Note that the degree of contact may be classified in accordance with an amount of movement of a treatment tool, for example. In an example case, the classification takes place in such a manner that the greater the amount of movement of the treatment tool is, the greater the degree of contact is.

The analysis function 542 performs an image analysis on endoscopic images sequentially acquired from the endoscopic system 2 to determine whether the treatment part is in contact with a blood vessel. The analysis function 542 calculates, when it is determined that the treatment part is in contact with a blood vessel, an amount of movement of the treatment part from a chronologically previous endoscopic image. The analysis function 542 then compares the calculated amount of movement with a classification in the determination conditions 531 to classify the degree of the contact. Note herein that the analysis function 542 detects an inducement event when the treatment part is in contact with a blood vessel, for example, and determines a classified degree of the contact as a risk level. For example, the analysis function 542 makes a determination in such a manner that the larger the degree of contact is, the higher the risk level is.

Furthermore, the condition “the degree of penetration of an electrosurgical knife into tissue” in the determination conditions 531 represents a condition when the degree of penetration of an electrosurgical knife is classified in a stepwise manner. Note that the degree of penetration may be classified in accordance with an amount of movement of an electrosurgical knife, for example. In an example case, the classification takes place in such a manner that the greater the amount of movement of an electrosurgical knife is, the greater the degree of penetration is. The analysis function 542 performs an image analysis on endoscopic images sequentially acquired from the endoscopic system 2. The analysis function 542 calculates an amount of movement of the electrosurgical knife from a chronologically previous endoscopic image. The analysis function 542 then compares the calculated amount of movement with a classification in the determination conditions 531 to classify the degree of the penetration. Note herein that the analysis function 542 detects an inducement event when a predetermined degree of penetration is exceeded, and determines a classified degree of the penetration as a risk level. For example, the analysis function 542 makes a determination in such a manner that the greater the degree of penetration is, the higher the risk level is.

Furthermore, the conditions “a period of time during which a blood vessel is pinched with forceps” and “a period of time during which an organ is held with forceps” in the determination conditions 531 each represent a condition when a period of time is classified in a stepwise manner. The analysis function 542 performs an image analysis on endoscopic images sequentially acquired from the endoscopic system 2. The analysis function 542 calculates “a period of time during which a blood vessel is pinched with forceps” and “a period of time during which an organ is held with forceps”. The analysis function 542 then compares each of the calculated periods of time with a classification in the determination conditions 531 to classify each of the calculated periods of time. Note herein that the analysis function 542 detects an inducement event when each of the calculated periods of time exceeds a predetermined period of time, and determines each of the periods of time as a risk level. For example, the analysis function 542 makes a determination in such a manner that the longer the calculated period of time is, the higher the risk level is.

Furthermore, the condition “the degree of deformation of an organ” in the determination conditions 531 represents a condition when the degree of deformation of an organ is classified in a stepwise manner. Note that the degree of deformation of an organ may be classified in accordance with an amount of change in shape of the organ, for example. In an example case, the classification takes place in such a manner that the greater the amount of change in shape of an organ is, the greater the degree of deformation is. The analysis function 542 performs an image analysis on endoscopic images sequentially acquired from the endoscopic system 2. The analysis function 542 calculates an amount of change in shape of the organ from a chronologically previous endoscopic image. The analysis function 542 then compares the calculated amount of change with a classification in the determination conditions 531 to classify the degree of the deformation. Note herein that the analysis function 542 detects an inducement event when a predetermined amount of change is exceeded, and determines a classified degree of the deformation as a risk level. For example, the analysis function 542 makes a determination in such a manner that the greater the degree of deformation is, the higher the risk level is.

Note that an organ deforms when the organ is held with forceps or when the organ is pulled with forceps, for example. Furthermore, an organ deforms when the insertion part of the endoscope 21 inserted into the body of a subject pushes the organ, for example.

Note that the image analysis described above may be performed along with a feature detection based on artificial intelligence (AI). Furthermore, in the example described above, an amount of movement of the treatment part and an amount of change of an organ are calculated through an image analysis. However, embodiments are not limited to the example described above. For example, when the position sensor 4 is used, position information acquired by the position sensor 4 may be used. In an example case, the analysis function 542 calculates an amount of movement of the treatment part, on the basis of position information acquired by the sensor part attached at a tip of the treatment part. Furthermore, for example, the analysis function 542 calculates an amount of deformation of an organ, on the basis of position information acquired by the sensor part disposed in the organ regarded as the involved part.

Furthermore, the analysis function 542 is able to detect other various types of events as events relating to abnormalities. In such a case, for example, determination conditions corresponding to an analysis using a medical image include conditions such as “whether there is a blood leak detected through color Doppler imaging”. For example, the condition “whether there is a blood leak detected through color Doppler imaging” in the determination conditions 531 represents a condition of whether a blood leak has occurred, and further represents a condition in which, when there is a blood leak, the degree of the blood leak is classified in a stepwise manner.

For example, the analysis function 542 performs an image analysis on color Doppler images sequentially acquired from an ultrasonic diagnostic apparatus representing the medical image diagnostic apparatus 1 to determine whether there is a blood leak. When the analysis function 542 determines that there is a blood leak, the analysis function 542 then detects the fact as an event relating to an abnormality. The analysis function 542 thus determines the degree of the blood leak as a risk level in accordance with the determination conditions 531. For example, the analysis function 542 makes a determination in such a manner that the greater the degree of a blood leak is, the higher the risk level is.

Furthermore, for example, determination conditions corresponding to an analysis using vital information include conditions such as “a reduction in blood pressure”, “an abnormality in electrocardiogram”, and “ischemia”. For example, the condition “a reduction in blood pressure” in the determination conditions 531 represents a condition of whether blood pressure is equal to or below a predetermined value, and further represents a condition in which, when there is such a reduction in blood pressure, the degree of the reduction in blood pressure is classified in a stepwise manner.

For example, the control function 541 acquires, from a blood pressure monitor, blood pressure information of the subject under surgery. The analysis function 542 determines, on the basis of the blood pressure information sequentially acquired from the blood pressure monitor, whether the blood pressure is equal to or below the predetermined value. When the analysis function 542 determines that the blood pressure is equal to or below the predetermined value, the analysis function 542 detects the fact as an event relating to an abnormality. The analysis function 542 then determines the degree of the reduction in blood pressure as a risk level in accordance with the determination conditions 531. For example, the analysis function 542 makes a determination in such a manner that the greater the degree of reduction in blood pressure is, the higher the risk level is.

Furthermore, for example, the condition “an abnormality in electrocardiogram” in the determination conditions 531 represents a condition of whether a rhythm or a waveform in an electrocardiogram has changed to exceed a predetermined amount of change, and further represents a condition in which, when there is such a change, the degree of the change is classified in a stepwise manner.

For example, the control function 541 acquires an electrocardiogram of the subject under surgery from an electrocardiograph. The analysis function 542 determines, on the basis of electrocardiograms sequentially acquired from the electrocardiograph, whether a rhythm or a waveform in the electrocardiograms has changed to exceed the predetermined amount of change. When a rhythm or a waveform in an electrocardiogram has changed to exceed the predetermined amount of change, the analysis function 542 detects the fact as an event relating to an abnormality. The analysis function 542 then determines the degree of the amount of the change as a risk level in accordance with the determination conditions 531. For example, the analysis function 542 makes a determination in such a manner that the greater the amount of change is, the higher the risk level is.

Furthermore, for example, the condition “ischemia” in the determination conditions 531 represents a condition of whether there is an ischemia, and further represents a condition in which, when there is an ischemia, the degree of the ischemia is classified in a stepwise manner. The analysis function 542 determines, on the basis of waveforms in electrocardiograms sequentially acquired from the electrocardiograph, whether there is an ischemia. When there is an ischemia, the analysis function 542 detects the fact as an event relating to an abnormality. The analysis function 542 then determines the degree of the ischemia as a risk level in accordance with the determination conditions 531. For example, the analysis function 542 makes a determination in such a manner that the greater the degree of an ischemia is, the higher the risk level is.

As described above, the analysis function 542 detects an event relating to an abnormality, on the basis of medical information acquired during surgery, and determines a risk level of the detected event relating to the abnormality. Note herein that the determination conditions described above are mere examples. An event relating to an abnormality may be detected on the basis of other types of information. For example, when a MEMS sensor is attached to the treatment part, and pressure information is acquired by the MEMS sensor, an event relating to an abnormality may be detected on the basis of the acquired pressure information.

In such a case, conditions relating to pressure information are stored as the determination conditions 531. For example, the condition “pressure” in the determination conditions 531 represents a condition of whether an acquired pressure value exceeds a predetermined value, and further represents a condition in which, when the value exceeds the predetermined value, the degree of the pressure is classified in a stepwise manner.

For example, the control function 541 acquires pressure information acquired by the MEMS sensor. The analysis function 542 determines, on the basis of the pressure information acquired by the control function 541, whether the pressure exceeds the predetermined value. When the analysis function 542 determines that the pressure exceeds the predetermined value, the analysis function 542 detects the fact as an event relating to an abnormality. The analysis function 542 then determines the degree of the pressure as a risk level in accordance with the determination conditions 531. For example, the analysis function 542 makes a determination in such a manner that the greater the pressure value is, the higher the risk level is.

The example analysis performed by the analysis function 542 has been described above. Note that, in the example described above, an event relating to an abnormality is detected on the basis of one of the conditions. However, embodiments are not limited to the example described above. An event relating to an abnormality may be detected on the basis of a plurality of combined ones of the conditions. For example, an event relating to an abnormality may be detected on the basis of a plurality of combined ones of the conditions (e.g., whether the treatment part is in contact with a blood vessel and the degree of deformation of an organ) for an analysis (e.g., an analysis using an endoscopic image) by a single device. Otherwise, an event relating to an abnormality may be detected on the basis of combined ones of the conditions for analyses (e.g., an analysis using an endoscopic image and an analysis using vital information) by a plurality of devices. Furthermore, various types of conditions in the determination conditions 531 to be referred to by the analysis function 542 may be set as desired. For example, it is possible to appropriately set such conditions in accordance with a type of a surgical operation and a type of an organ regarded as an involved part, for example.

The generation function 543 is configured to associate a point of time of detecting an event relating to an abnormality with medical information acquired at the point of time to generate association information. Specifically, the generation function 543 associates medical information when an event relating to an abnormality is detected by the analysis function 542 with a point of time of acquiring the medical information to generate association information. For example, when an event relating to an abnormality (an inducement event) is detected in an analysis based on the condition “the degree of deformation of an organ”, the generation function 543 associates an endoscopic image when the inducement event is detected with a point of time of capturing the endoscopic image to generate association information. Note that the point of time of capturing the endoscopic image is acquired simultaneously when the control function 541 acquires the endoscopic image.

Note herein that the generation function 543 may associate medical information when an event relating to an abnormality is detected with a time to generate association information. Otherwise, the generation function 543 may generate association information with which other pieces of medical information at the point of time of acquiring the medical information when an event relating to an abnormality is detected is further associated. For example, when vital information is acquired together with an endoscopic image, and an event relating to an abnormality is detected in an analysis based on the condition “the degree of deformation of an organ” using the endoscopic image, the generation function 543 is able to generate association information with which the vital information acquired simultaneously is further associated. That is, the generation function 543 generates association information with which vital information at a point of time of capturing the endoscopic image when an event relating to an abnormality is detected is further associated.

Furthermore, the generation function 543 is also able to associate, with a risk level, association information in which a point of time of acquiring medical information when an event relating to an abnormality is detected is associated with medical information acquired at the point of time. For example, the generation function 543 further associates association information with a risk level determined in an analysis based on the condition “the degree of deformation of an organ”.

Furthermore, the generation function 543 is also able to generate association information with which position information is associated. Specifically, the generation function 543 associates a point of time of detecting an event relating to an abnormality with medical information acquired at the point of time and position information acquired at the point of time to generate association information. In such a case, the sensor part of the position sensor 4 is first attached at the tip of the insertion part of the endoscope 21. Position information of the tip of the insertion part during surgery is then acquired. The control function 541 acquires, from the position sensor 4, position information acquired by the position sensor 4. The control function 541 associates the acquired position information with a point of time of acquiring the position information. The control function 541 then causes the storage circuitry 53 to store the position information associated with the point of time.

The generation function 543 acquires, from the storage circuitry 53, the position information at a point of time of acquiring the medical information when the event relating to the abnormality is detected by the analysis function 542. The generation function 543 then associates the point of time of acquiring the medical information when the event relating to the abnormality is detected with the medical information acquired at the point of time to generate association information. For example, when an event relating to an abnormality is detected in an analysis based on the condition “the degree of deformation of an organ”, the generation function 543 associates an endoscopic image when the event relating to the abnormality is detected with a point of time of capturing the endoscopic image and position information at the point of time to generate association information.

Note that it is possible to appropriately set information to be associated with association information. For example, the generation function 543 appropriately associates, in accordance with information acquired during surgery, as association information, a time, medical information, a result of analysis, and position information with each other.

Each time an event relating to an abnormality is detected by the analysis function 542 during surgery, the generation function 543 generates and causes the storage circuitry 53 to store association information as described above. The association information 532 in the storage circuitry 53 is generated and caused to be stored by the generation function 543, as described above. Note that the association information 532 may be stored per surgery, and may be read after surgery.

As described above, when the association information is generated, the control function 541 causes the display 52 to display various types of information using the association information. Furthermore, the control function 541 is also able to cause the display 22 in the endoscopic system 2 and the display 31 in the virtualized laparoscopic imaging system 3 to display various types of information using the association information.

Example information caused to be displayed by the control function 541 will now be described.

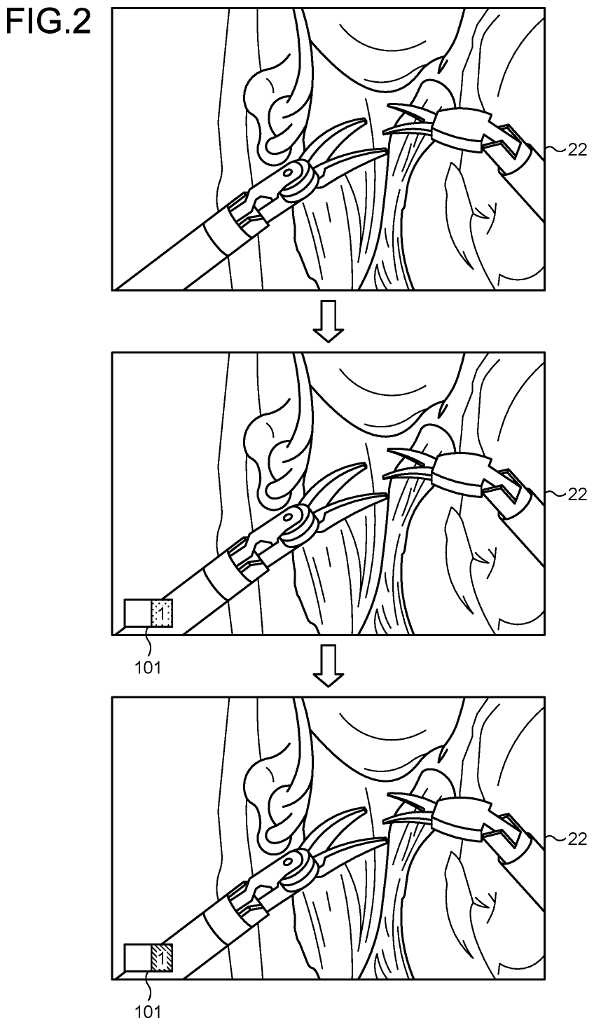

For example, the control function 541 causes time line information indicative of association information in a chronological order to be displayed. FIG. 2 is a view illustrating an example of display control executed by the control function 541 according to the first embodiment. Note herein that FIG. 2 illustrates displayed time line information with respect to an endoscopic image caused to be displayed by the endoscopic system 2.

For example, as illustrated at an upper portion in FIG. 2, in the endoscopic system 2, the display 22 is caused to display a real-time image of the inside of the abdominal cavity, on the basis of the endoscopic image acquired during surgery. Meanwhile, the surgery support apparatus 5 acquires various types of medical information to determine whether an event relating to an abnormality is occurring.

Note herein that, when the analysis function 542 detects an event relating to an abnormality, and the generation function 543 generates association information, the control function 541 performs control, as illustrated at a middle portion in FIG. 2, on the basis of the generated association information, to cause time line information 101 to be displayed in the endoscopic image.

Note herein that the control function 541 causes, as illustrated at a position 1 on the time line information 101, information identifying at least one of a level of the detected event relating to the abnormality or detection means to be displayed. For example, the control function 541 causes, at the position 1 on the time line information, in a colored manner in accordance with its risk level, a detected event relating to an abnormality to be displayed. Furthermore, for example, the control function 541 causes, at the position 1 on the time line information, in a colored manner in accordance with its detection means (e.g., conditions used for an analysis), a detected event relating to an abnormality to be displayed. Note herein that, to make a risk level and detection means identifiable, the control function 541 allocates a color to an outer frame and an inside color, at the position 1 on the time line information 101, in accordance with detection means and a risk level, respectively, for example. In an example case, the control function 541 causes the outer frame at the position 1 to be displayed in color corresponding to a color of detection means, and the inside of the frame at the position 1 to be displayed in color corresponding to a color of a risk level.

Furthermore, the control function 541 is able to make a change in display in accordance with a change of a risk level. As described above, the analysis function 542 detects an event relating to an abnormality, on the basis of medical information to be sequentially acquired by the control function 541. Therefore, when events relating to abnormalities are successively detected on the basis of medical information sequentially acquired, pieces of association information are successively generated. The control function 541 then causes the time line information to successively display information allowing the identification of such events.

Note herein that, for example, when the degree of deformation of an organ gradually changes, a risk level to be determined by the analysis function 542 gradually changes. In such a case, the risk level to be associated with association information to be generated by the generation function 543 changes. As a result, for example, the control function 541 causes, as illustrated at a lower portion in FIG. 2, a change in color at the position 1 on the time line information 101 to be occurred for display.

For example, the control function 541 does not cause the time line information 101 to be displayed as illustrated at the upper portion in FIG. 2, until association information is generated. When association information is generated, the control function 541 causes the time line information 101 to be automatically displayed as illustrated at the middle portion and the lower portion in FIG. 2. The control function 541 is also able to perform control to hide the time line information when the generation of association information is stopped.

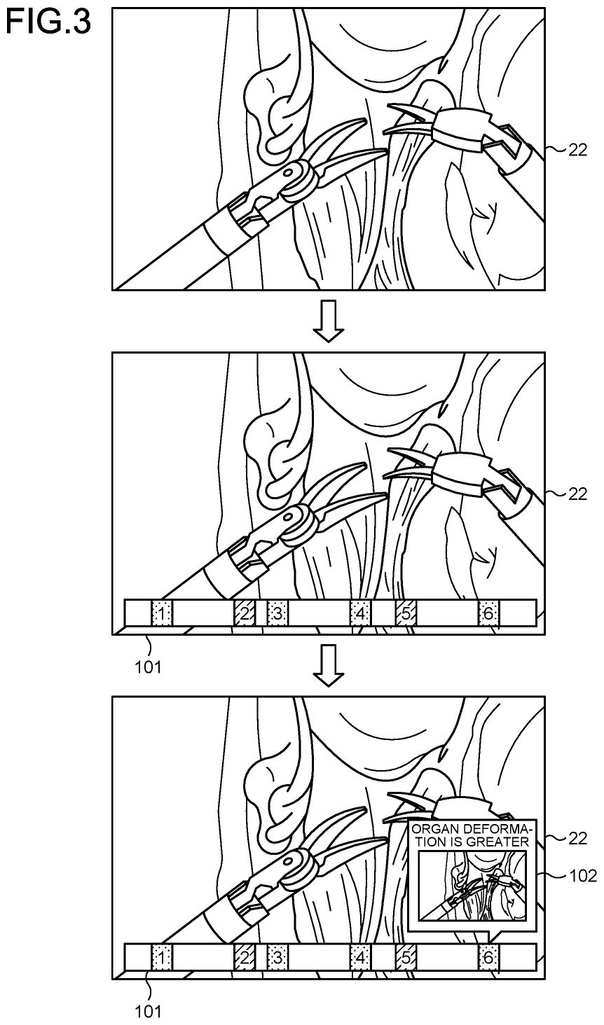

Furthermore, for example, the control function 541 is also able to cause a medical image collected, during surgery, at a point of time of detecting an event relating to an abnormality to be displayed in association with a corresponding point of time in the time line information. FIG. 3 is a view illustrating an example of the display control executed by the control function 541 according to the first embodiment. Note herein that FIG. 3 illustrates displayed time line information with respect to an endoscopic image caused to be displayed by the endoscopic system 2.

For example, the control function 541 follows an operation of an operator to display the time line information 101 and a thumbnail 102, as illustrated in FIG. 3. In an example case, when the operator executes a display operation for the time line information 101 during surgery, the control function 541 causes the time line information 101 based on association information generated so far to be displayed, as illustrated at a middle portion in FIG. 3.

Furthermore, when the operator makes a specification at a position 6 on the time line information 101, the control function 541 causes, as illustrated at a lower portion in FIG. 3, the thumbnail 102 of an endoscopic image corresponding to the specified position 6 to be displayed in association with the specification made at the position 6.

For example, when an abnormality occurs, such as bleeding, during surgery, and the operator causes the time line information to be displayed, the control function 541 causes the time line information 101 identifying a point of time of detecting an event relating to the abnormality to be displayed, as illustrated at the middle portion in FIG. 3. Therefore, the operator is able to easily identify the point of time of the occurrence of the event that is thought to be a cause of the abnormality.

When the operator then specifies a position on the time line information 101, the control function 541 causes, as illustrated at the lower portion in FIG. 3, the thumbnail 102 of an image acquired at a point of time of making the specification to be displayed. Therefore, the operator is able to know a treatment situation when an event relating to an abnormality occurred. The operator is also able to promptly identify a cause of an abnormality such as bleeding.

Furthermore, for example, the control function 541 causes, in a medical image of the subject, the position, corresponding to a point of time of detecting an event relating to an abnormality, of a medical apparatus to be displayed. That is, the control function 541 causes, on the basis of position information associated with association information, when the association information includes the position information, a medical image indicative of the position information acquired at a point of time of detecting an event relating to an abnormality to be displayed.

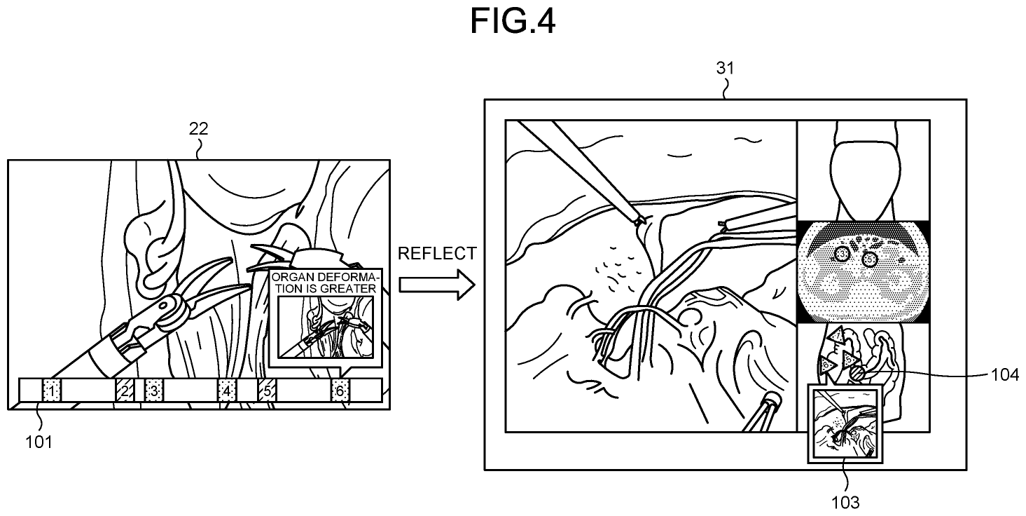

FIG. 4 is a view illustrating an example of the display control executed by the control function 541 according to the first embodiment. Note herein that an illustration on the left side in FIG. 4 illustrates an endoscopic image indicative of the time line information 101. Furthermore, an illustration on the right side in FIG. 4 illustrates a medical image indicative of position information.

For example, the control function 541 causes, as illustrated in FIG. 4, the endoscopic image to indicate the time line information 101 and a medical image indicative of position information of a medical apparatus at a point of time of generating association information. Note herein that the control function 541 uses a three-dimensional medical image for which an alignment has been executed with three-dimensional coordinates in a space where the magnetic field generation part in the position sensor 4 serves as an origin to cause a medical image indicative of position information to be displayed.

For example, the control function 541 uses, as illustrated on the right side in FIG. 4, a virtualized laparoscopic image based on a three-dimensional medical image for which an alignment has been executed with three-dimensional coordinates in a space where the magnetic field generation part serves as an origin, and uses a CT image to cause position information to be displayed. In an example case, the control function 541 identifies each position, in the three-dimensional medical image, corresponding to each piece of position information, on the basis of pieces of position information corresponding to points of time at the positions 1 to 6 on the time line information 101, and of alignment information.

The control function 541 then causes a medical image indicative of identification information at each identified position to be displayed. For example, the control function 541 causes, as illustrated in FIG. 4, numbers corresponding to the positions 1 to 6 on the time line information 101 to be displayed at identified positions on the CT image. Note herein that the control function 541 is able to cause each number to be displayed, similar to the time line information 101, allowing a detected risk level and detection means to be identified. For example, the control function 541 causes a risk level and detection means to be displayed in an identifiable manner by changing the shape of the outer frame surrounding a number and the inside color in the shape, for example.

Furthermore, the control function 541 is able to cause not only position information of a medical apparatus at a point of time of acquiring medical information when an event relating to an abnormality is detected, but also, in a medical image of the subject, the current position of the medical device (the tip of the endoscope 21) to be displayed. That is, the control function 541 identifies, in a three-dimensional medical image, a position, corresponding to the current position, of a medical device, when the position is acquired from the position sensor 4. The control function 541 then causes a medical image indicative of identification information at the identified position to be displayed. For example, the control function 541 causes a current position 104 of a medical device to be displayed, as illustrated on the right side in FIG. 4.

Furthermore, the control function 541 is able to follow an operation of the operator to cause a thumbnail 103 to be displayed, as illustrated in FIG. 4. In an example case, when the operator executes, during surgery, a display operation for the time line information 101 and a specification operation at the position 6 on the time line information 101, the control function 541 causes, as illustrated in FIG. 4, a virtualized laparoscopic image to reflect information of the specified position 6. The control function 541 further causes the thumbnail 103, corresponding to a time at the position 6, of the virtualized laparoscopic image to be displayed in association with the number.

For example, when an abnormality occurred, such as bleeding, during surgery, the control function 541 causes an image indicative of the position of a medical device at a point of time of acquiring medical information when an event relating to the abnormality is detected to be displayed. Therefore, the operator is able to easily identify the position at which the event that is thought to be a cause of the abnormality occurred.

When the operator then makes a specification at a position on the time line information 101, the control function 541 causes, as illustrated at the lower portion in FIG. 3, the thumbnail 103 of an image at a point of time of making the specification to be displayed. Therefore, the operator is able to know a treatment situation when an event relating to an abnormality occurred. The operator is also able to promptly identify a cause of an abnormality such as bleeding.

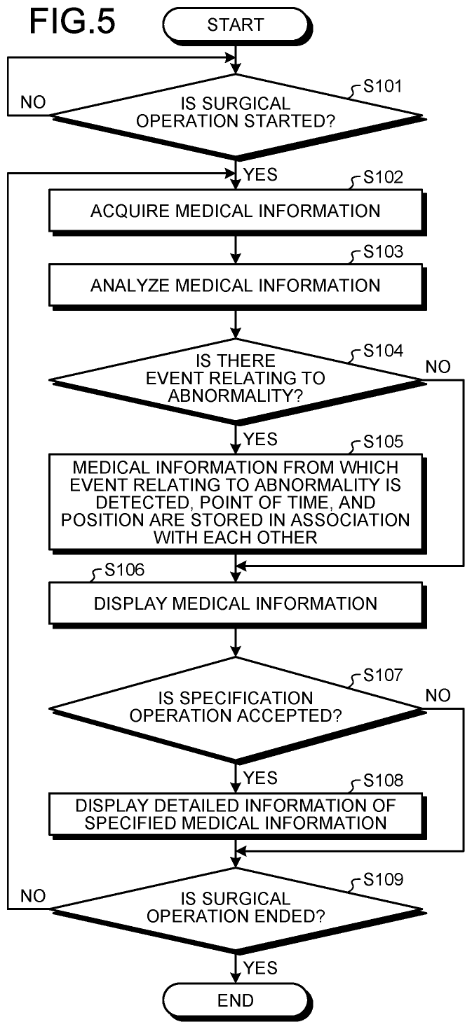

Next, processing performed by the surgery support apparatus 5 according to the first embodiment will be described with reference to FIG. 5. FIG. 5 is a flowchart for describing steps of processing executed by the surgery support apparatus 5 according to the first embodiment. Note that FIG. 5 illustrates an example case in which a time, medical information, and position information are associated with each other to serve as association information.

Note herein that steps S101, S102, and S106 to S109 in FIG. 5 are steps implemented when the processing circuitry 54 reads and executes a computer program corresponding to the control function 541 from the storage circuitry 53. Furthermore, step S103 in FIG. 5 is a step implemented when the processing circuitry 54 reads and executes a computer program corresponding to the analysis function 542 from the storage circuitry 53. Furthermore, steps S104 and S105 in FIG. 5 are steps implemented when the processing circuitry 54 reads and executes a computer program corresponding to the generation function 543 from the storage circuitry 53.

As illustrated in FIG. 5, in the surgery support apparatus 5, the processing circuitry 54 first determines whether a surgical operation is started (at step S101). For example, the processing circuitry 54 determines whether the surgical operation is started, on the basis of whether an operation of starting the processing is accepted. Note herein that, when the surgical operation is started (positive at step S101), the processing circuitry 54 acquires medical information (at step S102). Note that, before the surgical operation is started, the surgery support apparatus 5 is in a standby state (negative at step S101).

The processing circuitry 54 then analyzes the medical information (at step S103) to determine whether there is an event relating to an abnormality (at step S104). Note herein that, when there is an event relating to an abnormality (positive at step S104), the processing circuitry 54 associates medical information when the event relating to the abnormality is detected, a time, and position information with each other to generate association information and cause the association information to be stored (at step S105). On the other hand, at step S104, when no event relating to an abnormality is detected (negative at step S104), the processing circuitry 54 proceeds to step S106.

At step S106, the processing circuitry 54 then causes medical information to be displayed. For example, the processing circuitry 54 causes an endoscopic image to be displayed. Note herein that, for example, when association information is generated, the processing circuitry 54 causes an endoscopic image including time line information based on the association information to be displayed. The processing circuitry 54 then determines whether a specification operation is accepted (at step S107).

Note herein that, when a specification operation is accepted (positive at step S107), the processing circuitry 54 causes detailed information (e.g., a thumbnail) of medical information for which the specification is made to be displayed (at step S108). The processing circuitry 54 then determines whether the surgical operation is ended (at step S109). On the other hand, when no specification operation is accepted (negative at step S107), the processing circuitry 54 determines whether the surgical operation is ended (at step S109).

At step S109, when the surgical operation is not ended (negative at step S109), the processing circuitry 54 returns to step S102 to continuously acquire medical information. On the other hand, when the surgical operation is ended (positive at step S109), the processing circuitry 54 ends the processing.

As described above, according to the first embodiment, the control function 541 acquires medical information of a subject under surgery. The analysis function 542 is configured to detect an event relating to an abnormality, on the basis of the acquired medical information of the subject. The generation function 543 is configured to associate a point of time of detecting an event relating to an abnormality with medical information acquired at the point of time to generate association information. Therefore, the surgery support apparatus 5 according to the first embodiment is able to provide information regarding a point of time of the occurrence of an event relating to an abnormality, allowing an easy confirmation of information regarding the abnormality occurred during surgery.

For example, even when bleeding occurs during endoscopic surgery, presenting a point of time of detecting an event relating to the abnormality (the bleeding) allows a prompt identification of a location of the bleeding. As a result, it is possible to continue the endoscopic surgery without requiring a shift to laparotomy. The feature contributes to improve the patient's quality of life (QOL) after surgery.

Furthermore, according to the first embodiment, the analysis function 542 detects an inducement event that induces an abnormality, on the basis of medical information of a subject. The generation function 543 associates a point of time of detecting the inducement event with medical information acquired at the point of time to generate association information. Therefore, the surgery support apparatus 5 according to the first embodiment allows an easy confirmation of an inducement event occurred during surgery.

Furthermore, according to the first embodiment, the control function 541 causes time line information indicative of association information in a chronological order to be displayed. Therefore, the surgery support apparatus 5 according to the first embodiment is able to provide information allowing an easy observation of a point of time of detecting an event relating to an abnormality.

Furthermore, according to the first embodiment, the control function 541 causes a medical image during surgery, the medical image being collected at a point of time of detecting an event relating to an abnormality, to be associated with a corresponding point of time in the time line information to display the medical image. Therefore, the surgery support apparatus 5 according to the first embodiment is able to present, instead of a real-time image, an image at a point of time of detecting an event relating to an abnormality, allowing a further prompt identification of a location of bleeding, for example.

Furthermore, according to the first embodiment, the control function 541 further acquires position information indicative of the position of a medical device used during surgery. The generation function 543 associates a point of time of detecting an abnormality with medical information acquired at the point of time and position information acquired at the point of time to generate association information. Therefore, the surgery support apparatus 5 according to the first embodiment is able to further present position information, allowing a further prompt identification of a location of bleeding, for example.

Furthermore, according to the first embodiment, the control function 541 acquires position information indicative of the position of a medical device operated inside the subject under surgery. Therefore, the surgery support apparatus 5 according to the first embodiment allows the acquisition of position information of a medical device inside a subject.

Furthermore, according to the first embodiment, the control function 541 further acquires a medical image of the subject. The control function 541 causes, in the medical image of the subject, the position, corresponding to a point of time of detecting an event relating to an abnormality, of a medical device to be displayed. Therefore, the surgery support apparatus 5 according to the first embodiment is able to display position information allowing an easier observation.

Furthermore, according to the first embodiment, the control function 541 causes, in the medical image of the subject, the current position of a medical device to be displayed. Therefore, the surgery support apparatus 5 according to the first embodiment allows easy understanding of a positional relation between the current position of a medical device and the position at a point of time of acquiring medical information when an event relating to an abnormality is detected.

Furthermore, according to the first embodiment, the control function 541 causes information identifying at least one of a level of a detected event relating to an abnormality or detection means to be displayed. Therefore, the surgery support apparatus 5 according to the first embodiment allows understanding at a glance of the degree of an event relating to an abnormality or detection means.

Furthermore, according to the first embodiment, the analysis function 542 detects an event relating to an abnormality, on the basis of an image feature amount, acquired during surgery, in an image of the subject. Therefore, the surgery support apparatus 5 according to the first embodiment is able to easily detect an event relating to an abnormality.

### Other Embodiments

The first embodiment has been described so far. However, other various kinds of embodiments than the first embodiment described above may be implemented.

In the embodiment described above, the surgery support system 10 includes the surgery support apparatus 5. The surgery support apparatus 5 executes various types of processing. However, embodiments are not limited to the embodiment described above. The various types of processing of the surgery support method according to the present application may be executed in a single apparatus in the surgery support system 10. The various types of processing may otherwise be executed in a dispersed manner among a plurality of apparatuses in the surgery support system 10.

For example, analysis processing for a piece of medical information may be executed by a device that has acquired the piece of medical information. For example, the endoscopic system 2 may analyze an endoscopic image described above. In such a case, the processing circuitry 23 executes the analysis function 542 described above to detect an event relating to an abnormality, on the basis of the endoscopic image. Furthermore, an ultrasonic diagnostic apparatus may detect an event relating to an abnormality, on the basis of an ultrasonic image. A device such as an electrocardiograph may detect an event relating to an abnormality, on the basis of vital information.

Furthermore, generation processing for association information and various types of display processing regarding association information may be executed in the virtualized laparoscopic imaging system 3.

Furthermore, in the embodiment described above, the surgery support method according to the present application is applied to laparoscopic surgery. However, embodiments are not limited to the embodiment described above. The surgery support method according to the present application may be applied to other various types of surgery.

Note that, in the embodiment described above, the example case has been described, in which the acquisition unit, the detection unit, the generation unit, and the display control unit according to the present specification are respectively implemented by the control function, the analysis function, the generation function, and the control function of the processing circuitry. However, embodiments are not limited to the example case. For example, in addition to the implementation of the acquisition unit, the detection unit, the generation unit, and the display control unit according to the present specification with the control function, the analysis function, the generation function, and the control function according to the embodiment described above, the functions may be implemented by hardware, software, or a combination of hardware and software.

Furthermore, the term “processor” used in the embodiment described above means, for example, circuitry such as a central processing unit (CPU), a graphics processing unit (GPU), an application specific integrated circuit (ASIC), or a programmable logic device (e.g., a simple programmable logic device (SPLD), a complex programmable logic device (CPLD), or a field programmable gate array (FPGA)). Note herein that, instead of storing computer programs in a storage circuit, such a configuration may be applied that computer programs are directly incorporated in a circuit in a processor. In this case, the processor reads and executes the computer programs incorporated in the circuit to implement the functions. Furthermore, the processors according to the present embodiment are not each limited to a single circuit configured per processor. A plurality of independent circuits may be combined to configure a single processor to implement the functions.

Note herein that the computer programs executed by the processors are provided in such a manner that the computer programs are incorporated beforehand in a storage circuit such as read only memory (ROM), for example. Note that the computer programs may otherwise be provided in such a manner that the computer programs in the form of files in a format installable to the devices or in an executable format are recorded in a computer-readable, non-transitory storage medium such as a compact disc-read only memory (CD-ROM), a flexible disk (FD), a compact disc-recordable (CD-R), or a digital versatile disc (DVD). Furthermore, the computer programs may be stored on a computer coupled to a network such as the Internet, downloaded via the network, and provided or distributed. For example, the computer programs each include modules serving as the processing functions as described above. In actual hardware, as a CPU reads, from a storage medium such as a ROM, and executes each of the computer programs, the modules are loaded on a main storage device, and then generated on the main storage device.

Furthermore, the above components of the apparatuses according to the embodiment and the modifications are functionally and schematically illustrated, and may not be necessarily physically configured as illustrated. That is, a specific, dispersed or integrated form of the apparatuses is not limited to the forms illustrated in the embodiments. The apparatuses may be wholly or partially and functionally or physically configured in a dispersed or integrated manner in terms of a desired unit in accordance with various kinds of loads and use situations, for example. Furthermore, the processing functions implemented in the apparatuses may be wholly or partially implemented as desired through a CPU and a computer program analyzed and executed by the CPU, or implemented as wired logic hardware.

Furthermore, among the steps of the processing described above in the embodiment and the modifications, it is possible to execute manually some or all of the steps of the processing that has been described to be executed automatically. Otherwise, it is possible to execute automatically, with a known method, some or all of the steps of the processing that has been described to be executed manually. In addition, unless otherwise specifically described, it is possible to alter as desired the steps of processing and control, specific names, and information including various types of data and parameters described above in the specification and the accompanying drawings.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A surgery support system comprising processing circuitry configured to
acquire medical information of a subject under surgery,
detect an event relating to an abnormality, based on the acquired medical information of the subject, and
associate a point of time of detecting the event relating to the abnormality with the medical information acquired at the point of time to generate association information.

2. The surgery support system according to claim 1, wherein the processing circuitry is configured to
detect an inducement event that induces the abnormality, based on the medical information of the subject, and
associate a point of time of detecting the inducement event with the medical information acquired at the point of time to generate association information.

3. The surgery support system according to claim 1, wherein the processing circuitry is configured to cause time line information indicative of the association information in a chronological order to be displayed.

4. The surgery support system according to claim 3, wherein the processing circuitry is configured to cause a medical image during surgery, the medical image being collected at the point of time of detecting the event relating to the abnormality, to be displayed in association with a corresponding point of time in the time line information.

5. The surgery support system according to claim 1, wherein the processing circuitry is further configured to
acquire position information indicative of a position of a medical device used during surgery, and
associate the point of time of detecting the event relating to the abnormality with the medical information acquired at the point of time and the position information acquired at the point of time to generate association information.

6. The surgery support system according to claim 5, wherein the processing circuitry is configured to acquire position information indicative of a position of a medical device operated inside the subject during surgery.

7. The surgery support system according to claim 5, wherein the processing circuitry is further configured to
acquire a medical image of the subject, and
cause, in the medical image of the subject, a position of the medical device to be displayed, the position corresponding to the point of time of detecting the event relating to the abnormality.

8. The surgery support system according to claim 7, wherein the processing circuitry is configured to cause, in the medical image of the subject, a current position of the medical device to be displayed.

9. The surgery support system according to claim 3, wherein the processing circuitry is configured to cause information identifying at least one of a level of a detected event relating to an abnormality or detection means to be displayed.

10. The surgery support system according to claim 1, wherein the processing circuitry is configured to detect the event relating to the abnormality, based on an image feature amount in an image of the subject, the image being acquired during surgery.

11. A surgery support method comprising:
acquiring medical information of a subject under surgery;
detecting an event relating to an abnormality, based on the acquired medical information of the subject; and
associating a point of time of detecting the event relating to the abnormality with the medical information acquired at the point of time to generate association information.
